(19) United States
(12) Patent Application Publication
(10) Pub. No.: US 2004/0022771 A1
(43) Pub. Date: Feb. 5, 2004

(54) Transfer of cells, tissue, and other substances to bone

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/630,016

(22) Filed: Jul. 30, 2003

Related U.S. Application Data
(60) Provisional application No. 60/399,597, filed on Jul. 30, 2002.

(51) Int. Cl. A61K 45/00

(52) U.S. Cl. 424/93.7

(57) ABSTRACT

Human or animal bones are used to house and nurture other useful tissues. Once inside the bones, the transferred cells may become attached to the bone, and the blood vessels within the bone provide nutrition. The transferred cells or tissues can be injected as a solution of individual cells or small clumps of cells. Large numbers of cells supported with an extracellular matrix scaffold can be surgically implanted or injected. The extracellular matrix can be synthetic, resorbing, or derived from biologic sources. To assist with transfer, the cells or tissue may be contained in a bag before being placed in the bone. The non-bone materials may either be removed from the bone following sufficient growth and nourishment, or the biologic materials may remain in place to provide one or more desirable functions. For example, a bone's access to the blood stream should help the islet cells monitor blood glucose, and release insulin into the blood stream.

### REFERENCE TO RELATED APPLICATION

This application claims priority from U.S. Provisional Patent Application Serial No. 60/399,597, filed Jul. 30, 2002, the entire content of which is incorporated herein by reference.

### FIELD OF THE INVENTION

This invention relates generally to cell culture and, in particular, to the implantation of non-bone tissue into bone to promote the growth of useful biologic materials.

### BACKGROUND OF THE INVENTION

The bones of the human body serve many functions. In addition to providing support and protection of the body's soft tissues, many bones of the skeletal system also aid in the production of blood. The inner, cancellous, portion of bone contains marrow that houses the cells that reproduce to form the blood elements.

The structure of bones aids in the production of blood cells. Progenititor blood cells housed in bones are protected by the hard bones. The vascular anatomy of bones aids the release of cells into the blood stream. Arteries bring blood to bone cells and veins carry blood away. A rich vascular network within bones supplies the cells within the blood with oxygen and nutrients. Such facts are well known to orthopedic surgeons and most general physicians.

Surgical procedures and trauma to bones can result in life threatening blood loss. The anatomic features of bones that aid in the housing of progenititor blood cells also are beneficial to other ceils and organisms. For example, cancer cells often metastasize to bone. Similarly, bacteria that enter the blood stream may reproduce within the bone, causing a pathologic condition known as osteomyelitis.

### SUMMARY OF THE INVENTION

Broadly, this invention uses the features of bone to house and nurture other useful tissues. The invention could be used to transfer living cells of any appropriate type that do not reside within bone under normal conditions, to bones of the human body.

In the preferred embodiment, non-bone cells are injected or surgically implanted into one or more bones of a patient. Once inside the bones, the transferred cells may become attached to the bone, and the blood vessels within the bone provide nutrition. The transferred cells or tissues can be injected as a solution of individual cells or small clumps of cells. Large numbers of cells supported with an extracellular matrix scaffold can be surgically implanted or injected. The extracellular matrix can be synthetic, resorbing, or derived from biologic sources.

To assist with transfer, the cells or tissue may be contained in a bag before being placed in the bone. Alternatively, a collapsed bag could be inserted into the bone then filled following placement. The bag is preferably porous, with holes sized to prevent cells form escaping. The pores may be dimensioned to inhibit the ingress or egress of all cells, or to allow migration of red blood cells in and out of the transferred tissue, while prohibiting migration of the transferred cells. Alternatively, the enclosure could have pores dimensioned to allow vessels to grow in to the transferred cells or tissue.

The invention offers numerous important advantages. The non-bone materials may either be removed from the bone following sufficient growth and nourishment, or the biologic materials may remain in place to provide one or more desirable functions. For example, a bone's access to the blood stream should help the islet cells monitor blood glucose, and release insulin into the blood stream.

### BRIEF DESCRIPTION OF THE DRAWINGS

FIG. 1A is an anterior view of the tibia and fibula;

FIG. 1B is an anterior view of a bag used in one embodiment of the invention;

FIG. 1C is an anterior view of the tibia and fibula; and

FIG. 1D is an anterior view of the tibia and fibula after placement of the transferred cells or transferred tissue.

### DETAILED DESCRIPTION OF THE INVENTION

Broadly, this invention uses the features of bone to support and house other useful tissues. The invention could be used to transfer living cells of any appropriate type that do not reside within bone under normal conditions, to bones of the human body.

In the preferred embodiment, non-bone cells are injected or surgically implanted into one or more bones of a patient. Once inside the bones, the transferred cells may become attached to the bone, and the blood vessels within the bone provide nutrition to the transferred cells. The transferred cells can be injected as a solution of individual cells or small clumps of cells. Large numbers of cells supported with an extracellular matrix scaffold can be surgically implanted or injected. The extracellular matrix can be synthetic, resorbing, or derived from biologic sources.

The invention has many uses, including the treatment of patients with diabetes. Researchers have experimented with injecting islet cells from the pancreas into the abdomen of patients with diabetes. Once inside the abdomen, a number of cells survive by adhering to the peritoneum. The surviving islet cells can produce functional insulin.

For the reasons listed above, bone should provide a better host to the islet cells of the pancreas than the peritoneum. The rich blood supply of bones would aid in the support of islet cells and in regulation of blood glucose. A bone's access to the blood stream would help the islet cells monitor blood glucose, and release insulin into the blood stream.

In the embodiment above, islet cells preferably harvested from the pancreas of human donors are grown in tissue culture using techniques well known to those skilled in that art. The cultured islet cells, perhaps including the extra cellular matrix that supports them, are injected into the vertebrae of the diabetic patient's spine through a large bore cannula.

The islet cells are ideally harvested from live, closely genetically related humans, such as siblings. Alternatively, the cells could be harvested from recently deceased humans, animals, or fetuses, including stem cells. The cells could also be transferred to other bones such as the bones of the pelvis. Additional factors, such as tissue growth factors, factors that promote vessel growth, differentiation factors, tissue culture, media, immunosuppressive medications, or antibiotics could be added.

To assist with transfer, the cells or tissue may be contained in a bag before being placed in the bone. Alternatively, a collapsed bag could be inserted into the bone then filled following placement. The bag is preferably porous, with holes sized to prevent cells form escaping. The manufacture of suitable cell or tissue enclosures is well known to those skilled in the art. As one example, U.S. Pat. No. 4,904,260 describes a method of manufacturing a suitable enclosure. Pores between 1-7 micrometers would inhibit the ingress or egress of all cells. Larger pores would allow migration of red blood cells in and out of the transferred tissue, while prohibiting migration of the transferred cells. Alternatively, the enclosure could have pores large enough to allow vessels to grow in to the transferred cells or tissue.

As discussed in U.S. patent application Ser. No. 09/897,000, incorporated herein by reference, many important therapeutic or bioactive materials may be added to the transferred cells. For example, Vascular Endothelial Growth Factor (VEGF) could be added to stimulate vessels to grow into the transferred cells or tissue. The therapeutic substances could be contained in a separate semi-porous bladder that is placed adjacent to the transferred cells. The '260 describes a suitable semi-porous bladder. Alternatively, the therapeutic substances could be released from a resorbable material. For example, the therapeutic substances could be released from an implanted hydrogel. Suitable hydrogels are well known to those skilled in the art.

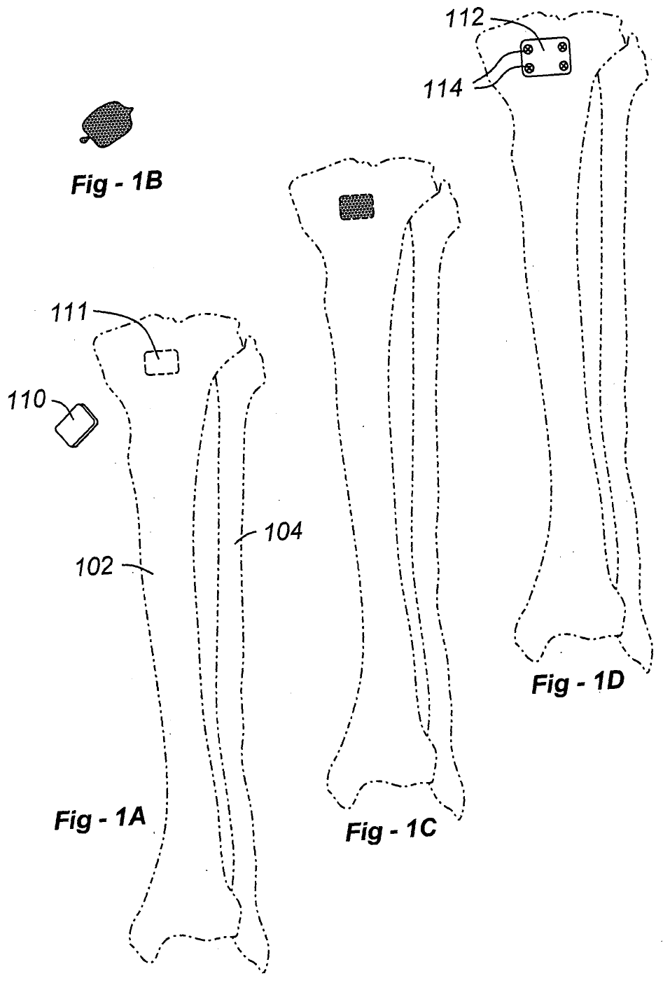

The transfer may occur into any suitable host bone section though, in the preferred embodiment, long bones from the leg are perhaps most convenient. FIG. 1A is an anterior view of the tibia 102 and fibula 104, and wherein a rectangular section of bone 110 has been removed from the anterior surface of the tibia. The resulting bone window 111 allows access to the intramedullary portion of the tibia. The transferred cells or transferred tissue is inserted into the intramedullary portion of the bone.

FIG. 1B is an anterior view of a bag useful in transferring cells or transferred tissue enclosed within the bag. FIG. 1C is an anterior view of the tibia and fibula with the bag described in FIG. 1B having been placed into the tibia. FIG. 1D is an anterior view of the tibia and fibula after placement of the transferred cells or transferred tissue. The removed section of tibia has been returned to the tibia. A plate 112 and screws 114 could be used to hold the piece of bone and/or transferred cells or transferred tissue, within the bone.

I claim:

1. A method of developing biologic material, comprising the steps of:
injecting or surgically implanting non-bone cells or tissue into one or more bones of a vertebrate; and
benefiting from the non-bone cells or tissue after receiving nourishment from the blood vessels within the bone.

2. The method of claim 1, wherein the step of injecting or surgically implanting the non-bone cells or tissue includes the transfer of individual cells or small clumps of cells.

3. The method of claim 1, further including the step of injecting or surgically implanting an extracellular matrix scaffold in conjunction with the non-bone cells or tissue.

4. The method of claim 3, wherein the extracellular matrix is synthetic, resorbing, or derived from biologic sources.

5. The method of claim 1, including the step of injecting or surgically implanting islet cells.

6. The method of claim 5, including the step of injecting or surgically implanting the extracellular matrix that supports the islet cells.

7. The method of claim 5, including the step of injecting or surgically implanting the islet cells into the vertebrae of a recipient.

8. The method of claim 5, wherein the islet cells are harvested from the pancreas of a human or animal donor.

9. The method of claim 8, wherein the human donor is the sibling of a recipient.

10. The method of claim 8, wherein the islet cells are harvested from a fetus in the form of stem cells.

11. The method of claim 5, including the step of injecting or surgically implanting the islet cells into the pelvis of a recipient.

12. The method of claim 1, including the step of injecting or surgically implanting one or more additional factors, such as tissue growth factors, factors that promote vessel growth, differentiation factors, tissue culture, media, immunosuppressive medications, or antibiotics.

13. The method of claim 1, including the step of placing non-bone cells or tissue into a porous bag for the purpose of implantation.

14. The method of claim 13, wherein the bag includes pores dimensioned to inhibit the ingress or egress of all cells.

15. The method of claim 13, wherein the bag includes pores dimensioned to allow migration of red blood cells in and out of the transferred tissue, while prohibiting migration of the transferred cells.

16. The method of claim 13, wherein the bag includes pores dimensioned to allow blood vessels to grow in to the transferred cells or tissue.

17. The method of claim 12, wherein the additional factors are contained in a separate, semi-porous enclosure adjacent to the transferred cells or tissue.

18. The method of claim 12, including an additional factor which is released from an implanted hydrogel or other resorbable material.

19. The method of claim 12, wherein the additional factors include Vascular Endothelial Growth Factor (VEGF) to stimulate vessels to grow into the transferred cells or tissue.

20. The method of claim 1, including the steps of:
removing a portion of a cortical bone layer to create a window into the cancellous region;
implanting the cells or tissue into the bone through the window; and
closing the window with a plate and screws or other seal.

21. The method of claim 20, wherein the window is created in a long bone.

22. The method of claim 20, wherein the window is created in a tibia or femur.
